(19) United States
(12) Patent Application Publication
(10) Pub. No.: US 2015/0197151 A1
(43) Pub. Date: Jul. 16, 2015

(54) Programmable Fuel Cell and Grommet Warm-Up Circuitry and Methods for Use in Sobriety Testing Systems

(71) Applicant: 1A Smart Start, Inc., Grapevine, TX (US)

(72) Inventor: James Ralph Ballard, JR., Flower Mound, TX (US)

(73) Assignee: 1A SMART START, INC., Grapevine, TX (US)

(21) Appl. No.: 14/593,012

(22) Filed: Jan. 9, 2015

Related U.S. Application Data
(60) Provisional application No. 61/927,628, filed on Jan. 15, 2014.

(51) Int. Cl. B60K 28/06; G01N 27/407; H04L 29/08; A61B 5/18

(52) U.S. Cl. CPC B60K 28/063; A61B 5/18; G01N 27/407; H04L 67/32; B60K 2028/003

(57) ABSTRACT

A subsystem for warming-up a substance sensor within a sobriety testing system includes a memory storing a schedule of times at which tests are expected to be taken by a user. Processing circuitry responsive to the stored schedule activates a heater associated with the substance sensor sufficiently in advance of an upcoming time on the schedule such that the substance sensor is at operating temperature when the corresponding test is requested by at the user.

### CROSS-REFERENCE TO RELATED APPLICATION

The present application claims the benefit of U.S. Provisional Patent Application Ser. No. 61/927,628, filed Jan. 15, 2014, which is incorporated herein by reference for all purposes.

### FIELD OF INVENTION

The present invention relates in general to sobriety testing techniques, and in particular to programmable substance sensors and grommet warm-up circuitry and methods for use in sobriety testing systems.

### BACKGROUND OF INVENTION

Sobriety testing, which includes testing for both alcohol and illegal drugs, has taken a prominent role in ensuring a safe and efficient society. For example, ignition interlocks on vehicles have proven their worth in preventing intoxicated drivers from entering the roadways and causing serious, including fatal, accidents. Sobriety testing has also allowed authorities, such as courts and law enforcement agencies, to monitor compliance with the court-ordered restrictions imposed on persons having committed alcohol or drug related offenses. Among other things, with the availability of reliable sobriety testing systems, such offenders can continue travel to work, school, or rehabilitation and thus contribute to society, rather than be a burden.

One particular type of sobriety testing system uses a fuel cell assembly to detect the presence of alcohol in the breath of a test subject. A typical fuel cell assembly includes a fuel cell, which implements a chemical reaction that produces an electrical current proportional to breath alcohol content, a grommet for receiving breath airflow from the test subject through an associated mouthpiece, and a pump for pulling a breath sample from the air flowing through the grommet into the fuel cell for testing. Given that the chemical reaction implemented by the fuel cell is normally temperature-sensitive, a typical fuel cell assembly will also include a fuel cell heater, for heating gas within the fuel cell itself, and/or a grommet heater, for heating the air passing through the grommet.

At cold or very cold temperatures, a fuel cell-based sobriety testing device must be warmed-up prior to administration of the test to ensure accurate results. Only after the testing device is at operating temperature and the breath test has been successfully passed is the vehicle allowed to start such that the passenger compartment heater can begin to operate. Depending on the severity of the cold environment within the passenger compartment, interlock warm-up can take several minutes, during which the driver test subject, as well as any passengers within the vehicle, are subjected to the same cold temperature. Even with suitable clothing, the cold temperature environment within the passenger compartment is, at a minimum, very uncomfortable. If children are accompanying the driver, the problems associated with the cold temperature environment are only compounded.

### SUMMARY OF INVENTION

According to one representative embodiment of the principles of the present invention, a subsystem is disclosed for warming-up a fuel cell within a sobriety testing system. A memory stores a schedule of times at which tests are expected to be taken by a user. Processing circuitry responsive to the stored schedule activates a heater associated with the fuel cell sufficiently in advance of an upcoming time on the schedule such that the fuel cell is at operating temperature when the corresponding test is requested by at the user.

The embodiments of the principles of the present invention provide numerous advantages, including the capability of having the fuel cell and grommet of a sobriety testing device to be automatically warmed to operating temperature and ready for use in advance of the driver entering the vehicle passenger compartment. As a result, the user, and any passengers accompanying the driver, are no longer required to sit within a cold or very cold environment for several minutes while waiting for the sobriety testing device to warm up. User and passenger comfort and safety are in turn enhanced.

In addition, the user is provided with significant flexibility in setting up a schedule of expected test times for warming the fuel cell and grommet in advance. For example, the user can directly program a schedule, through the user interface devices such as a keypad or microphone. Alternatively, adaptive learning allows the sobriety test unit to automatically record events and program a schedule based on statistics showing a pattern in the user's behavior. The principles of the present invention also account for variations in those patterns as the result of weekends and holidays where vehicle usage is expected to change.

### BRIEF DESCRIPTION OF DRAWINGS

For a more complete understanding of the present invention, and the advantages thereof, reference is now made to the following descriptions taken in conjunction with the accompanying drawings, in which:

FIG. 1A is a diagram of a portion of an interior of a vehicle including a sobriety interlock system suitable for demonstrating one possible application of the principles of the present invention;

FIG. 1B is a high level functional block diagram of the exemplary sobriety interlock system utilized in the application shown in FIG. 1A;

FIG. 2 is a more detailed functional block diagram showing the primary subsystems of the handheld unit shown in FIG. 1B;

FIG. 3 is a flow chart illustrating an exemplary procedure for automatic scheduling of sobriety interlock system fuel cell and grommet advance warm-up according to the principles of the present invention;

FIG. 4 is a flow chart illustrating an exemplary procedure for advance warm-up of a sobriety interlock system fuel cell and grommet according to the principles of the present invention;

FIG. 5 is a flow chart illustrating another exemplary procedure for advance warm-up of a sobriety interlock system fuel cell and grommet in a sobriety according to the principles of the present invention;

FIG. 6 is a flow chart illustrating an exemplary procedure for updating the warm-up schedule in the sobriety interlock system of FIGS. 1A and 1B; and

FIG. 7 is a flow chart illustrating an exemplary procedure for updating the warm-up schedule in a server operating in conjunction with the sobriety interlock system of FIGS. 1A and 1B.

### DETAILED DESCRIPTION OF THE INVENTION

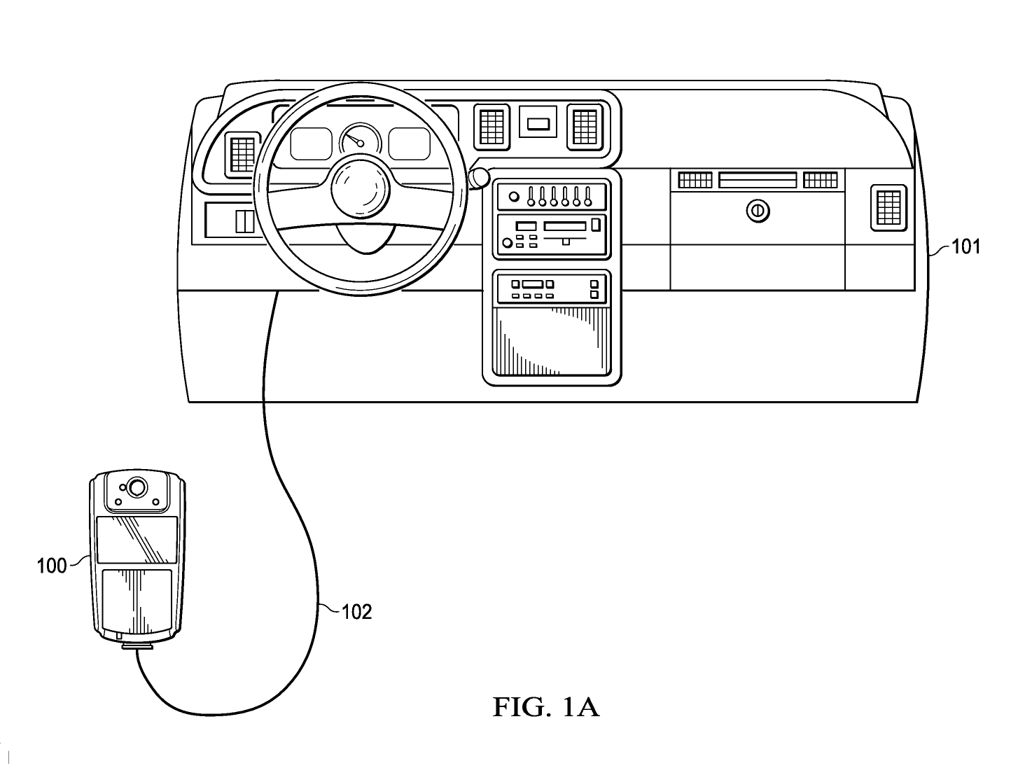

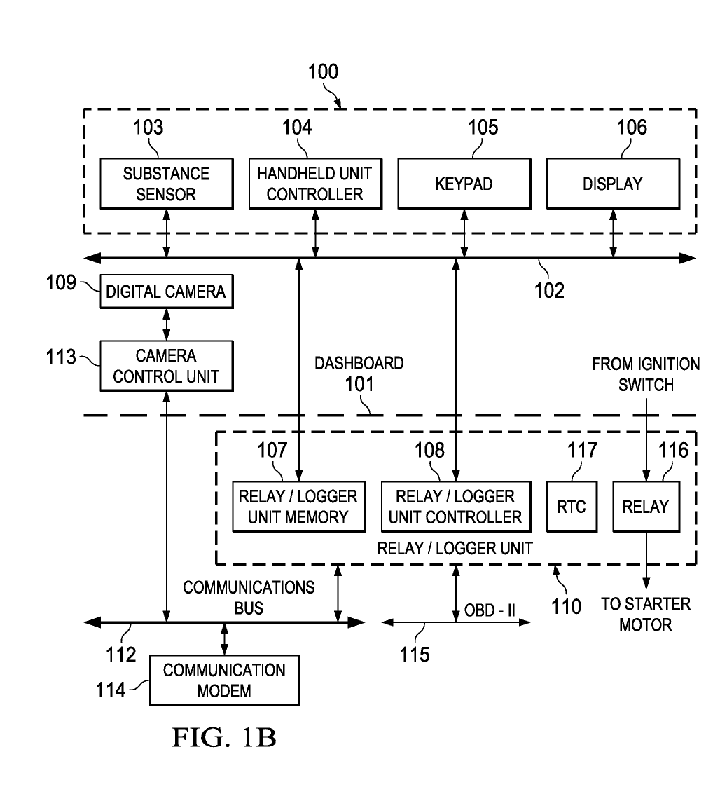

The principles of the present invention and their advantages are best understood by referring to the illustrated embodiment depicted in FIGS. 1-5 of the drawings, in which like numbers designate like parts. For discussion purposes, these principles will be described in conjunction with an alcohol breath testing system operating within an vehicle ignition interlock system. It should be recognized, however, that the systems and methods described below are equally applicable to other types of sobriety testing systems, including stand-alone sobriety testing systems and those designed to test for other types of intoxicants and controlled substances (e.g., marijuana).

FIG. 1A is a diagram showing a portion of the interior of a motor vehicle in the area of the dashboard. A handheld breath alcohol testing unit 100 is connected to electronic circuitry behind vehicle dashboard 101 (see FIG. 1B) through a cable 102. Generally, a person attempting to start the vehicle must provide a breath sample to handheld unit 100, which tests for deep-lung breath alcohol content, deep-lung alcohol content being directly proportional to blood alcohol concentration and thus intoxication level. If the person being tested passes the breath alcohol test, the interlock system allows the vehicle to start. On a test failure, the interlock system disables the vehicle ignition system and the vehicle is rendered inoperable.

FIG. 1B is a high level functional block diagram of the overall interlock system. Handheld unit 100, which is discussed in detail below, includes a substance sensor 103, which in the illustrated embodiment is a fuel cell alcohol sensor, a handheld unit controller 104, a keypad 105 for data entry, and a display 106.

Handheld unit 100 electrically communicates through cable 102 with electronics behind dashboard 101. The electronics behind dashboard 101 include relay/logger unit memory 107 and relay/logger unit controller 108. Relay/logger unit memory 107, which is preferably solid state memory, such as Flash memory, stores the results of tests performed by handheld unit 100 for periodic retrieval and review by authorities monitoring the driver for compliance with any conditions or restrictions imposed on the driver. In addition, relay/logger unit memory stores the firmware controlling the operation of relay/logger unit controller 108.

Relay/logger unit controller 108, operating in conjunction with handheld unit 100, controls the operation of the vehicle in response to the outcome of a given test. As known in the art, the ignition system of a vehicle can be controlled in any one of a number of ways, including enabling or disabling relays providing power to the starter motor or sending enable or disable commands to one or more on-board computers. In the illustrated embodiment, relay/logger unit controller 108 controls a relay 116, which in turn controls the flow of electrical current between the vehicle ignition switch and the starter motor. Relay/logger unit controller 108 may also be used to generate visible or audible warnings in the event of a failed test, for example, causing the horn to sound or the headlights to flash.

A digital camera 109 or similar imaging device is also preferably provided to allow for positive identification of the person taking the breath test through handheld unit 100. The images taken by digital camera 109 are preferably stored in relay/logger unit memory 107 for retrieval and review by the monitoring authorities. Advantageously, digital camera 109 reduces the possibility of a restricted or intoxicated driver of circumventing the interlock system by having a substitute person providing the breath sample to handheld unit 100. In the illustrated embodiment, digital camera 109 operates in conjunction with a camera control unit 113, which communicates with relay/logger unit controller 108 via an RS-485 standard bus 112.

Also operating off of RS-485 bus 112 is a cellular telecommunications modem 114, which allows relay/logger unit controller 108 to wireless send alerts to the authorities in the event of a failed test (i.e., the detection of a controlled substance) or transmit logged information within relay/logger unit memory 107 to the monitoring authorities, whether or not an intoxicated driver has been detected.

In one particular embodiment, handheld unit 100, relay/logger unit memory 107, relay/logger unit controller 108 communicate, either in whole or in part, with the OBD-II diagnostic system 115 standard on most motor vehicles. The OBD-II system provides another efficient mechanism by which monitoring authorities can access the data stored within relay/logger unit memory 107 through a standard OBD-II port and associated test equipment. In addition, the OBD-II also allows for vehicle operating data to be recorded and stored within relay/logger unit memory 107 for correlation with the results of sobriety testing performed through handheld unit 100.

The OBD-II diagnostic system also provides a communications path for transmission of command and control signals from relay/logger unit controller 108 to various electronics and electrical systems within the vehicle. These command and control signals can be used by interlock system controller 104 to disable the vehicle in response to a failed intoxication test.

In the illustrated embodiment, relay/logger unit controller 108 includes a microprocessor or microcontroller, such as a Renesas RSF3650KDFB or similar device. A real time clock 117, such as a Seiko S-35390Q, operating in conjunction with relay/logger unit controller 108, tracks the date and time.

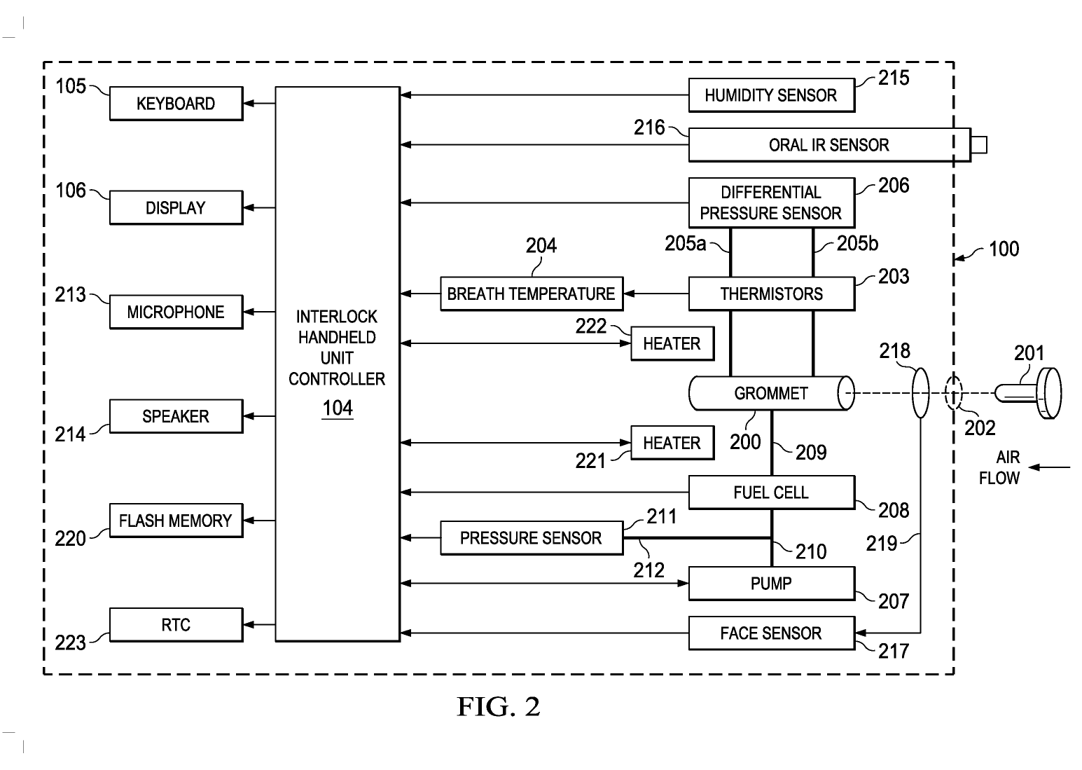

FIG. 2 is a more detailed functional block diagram of the primary subsystems within handheld unit 100 in a preferred embodiment of the principles of the present invention. In this embodiment, interlock system controller 104 is a Renesas RSF3650KDFB processor operating in conjunction with firmware stored in Flash memory 220. For clarity, interface devices, such as the analog to digital converters (ADCs) interfacing the various blocks with controller 104, and auxiliary subsystems, are not shown in FIG. 2.

A cylindrical grommet 200 receives a disposable mouthpiece 201 through an aperture 202 through the front panel of the case of handheld unit 100. Air introduced by a user (i.e., the human test subject) through mouthpiece 201 generally passes through cylindrical grommet 200 and passes out an aperture through the unit rear panel.

As air flow passes through grommet 200, a set of at least one thermistor 203 and associated breath temperature measurement circuitry 204 measure breath temperature. Breath temperature is one parameter useful for detecting attempts to circumvent an alcohol breath test.

A pair of tubes 205a-205b tap the airflow through grommet 200 to a differential pressure sensor 206, which measures breath pressure and breath air flow rate. As known in the art, in order for an alcohol breath test to be valid, the user must provide sufficient air pressure for a sufficiently long period of time to ensure that a deep-lung air is received by the alcohol sensor. If neither of these two conditions is met, interlock system controller 104 aborts the test and the breath test functional routine is reset. One device suitable for use as differential pressure sensor 206 in the embodiment of FIG. 2 is a Sensormatic 35AL-L50D-3210 differential pressure transducer.

Once interlock system controller 104 determines that deep-lung air is being received, a pump 207 is activated to draw a sample of the air flowing through grommet 200 into a fuel cell 208. In the illustrated embodiment, the air sample is drawn through tubes 209 and 210. A pressure sensor 211 monitors the air pressure being provided by pump 207 through a tube 212. One suitable fuel cell 208 is a Dart Sensors LTD 2-MS3 fuel cell operating in conjunction with a pump 207 available from PAS International, although other commercially available fuel cells and pumps may be used in alternative embodiments. A suitable device for pressure sensor 211 is a Sensormatic 33AL-L50D-3210 pressure transducer.

Fuel cell 207 implements a well-known electrochemical process to determine the breath alcohol content of the deep-lung air sample. From the air sample, interlock system controller 104 calculates the corresponding blood alcohol concentration and determines whether the user has passed or failed the test, depending on the legal limits imposed by the given jurisdiction. In response to the test result, interlock system controller 104 sends commands to vehicle electronics/electrical system 108 to enable or disable the vehicle ignition system. The results of the test are also recorded within relay/logger unit memory 107 for access by the monitoring authorities.

The user interacts with system controller 104 through keypad 105 and display 106, which allow the user to receive prompts and initiate a test in anticipation of starting the vehicle. In addition, interlock system controller 104 may periodically require retest of the user to ensure driver sobriety after initial start of the vehicle. In alternate embodiments, a microphone 213 and speaker 214 allow for control of handheld unit 100 by voice command.

In the illustrated embodiment of handheld unit 100, multiple sensors are provided for preventing circumvention of the breath test. In addition to breath temperature circuitry 204, handheld unit 100 also includes a humidity sensor 215, an oral infrared (IR) sensor 216, and a face proximity sensor 217. In the embodiment shown in FIG. 2, face proximity sensor 217 operates in conjunction with an electrode 218 disposed on the inner surface of the front panel of the case of handheld unit 100 and at least partially surrounding aperture 202. A clip 219 provides an electrical connection between the printed circuit board on which face proximity sensor circuit 217 resides and electrode 218.

Temperature can have a significant effect on the operation of handheld unit 100 at cold or very cold temperatures. Among other things, the speed of the electrochemical reaction within fuel cell 208 typically decreases with decreasing temperature. In addition, fuel cell 208 also is subject to a temperature coefficient, wherein the strength of the generated detection signal decreases with decreasing temperature. In addition, when grommet 200 is cold, condensation from the test subject's breath can adversely impact the test measurement.

In order to ensure proper breath content measurements are taken, grommet 200 is heated by a heater 222, which is, for example, one or more metallic sheets disposed around the grommet outer periphery. Similarly, a heater 221 maintains the temperature of fuel cell 208. Heater 221 may be, for example, a metallic sheet disposed against one or more of the outer surfaces of fuel cell 208 or a metal block on which fuel cell 208 sits. In embodiments of handheld unit 100 using a Renesas R5F3650NDFB microcomputer, heaters 221 and 222 are driven with pulse width modulated (PWM) signals that can be made available at certain controller input/output pins by firmware programming. In addition, the temperature of fuel cell heater 221 and grommet heater 222 are monitored and corresponding signals returned to handheld unit controller 104.

At cold or very cold temperatures, heaters 221 and 222 may require several minutes to bring grommet 200 and fuel cell 208 up to operating temperature. As discussed above, during this period, the occupants of the vehicle are subject to the existing cold temperature environment within the vehicle passenger compartment. Hence, according to the principles of the present invention, warm-up of grommet 200 and fuel cell 208 in advance of expected tests is scheduled by date and time. The particular schedule can be programmed into the interlock system by the user, for example using keyboard/keypad 105 and display 106 or by automatic adaptive learning using statistical observation of the user's behavior.

In the two-controller system discussed above, the programming and control of the warm-up process can be implemented in a different way, which advantageously allows for optimization in the use of available processing resources. In one embodiment, while the vehicle is shut-down, handheld unit 100 is powered-down and at least parts of the relay/logger unit, including RTC 117 and relay/logger unit controller 108, remain powered-up. In this case, the warm-up schedule is programmed into relay/logger unit memory 107. As the expected time and day for the test approaches, as measured by RTC 117 and/or internally by the controller 108, relay/logger unit controller 108 provides power to handheld unit 100 sufficiently in advance to allow handheld unit controller 104 to activate heaters 221 and 222 and bring grommet 200 and fuel cell 204 to their operating temperatures before the expected test time.

In a second embodiment, while the vehicle is shut-down, handheld unit 100 remains at least partially powered up and RTC 223 tracks the day and time. In this embodiment, the schedule is programmed into handheld unit FLASH memory 220. As the expected time and day for the test approaches, as measured by RTC 223, handheld unit controller 104 activates heaters 221 and 222 and brings grommet 200 and fuel cell 208 to their operating temperatures before the expected test time.

In a third embodiment, the programming and control functions are split between relay/logger unit memory 107, handheld unit FLASH memory 220, relay logger unit controller 108, and handheld unit controller 104. In the illustrated embodiment, the use of compatible controller, a common bus, and FLASH memory in both the handheld unit and the relay/logger unit provide options to the system programmer for best allocating available processing resources to the tasks required to implement scheduled warm-up in view of the other processing tasks that must be performed. For example, the required firmware could be split between relay/logger unit memory 107 and handheld unit FLASH memory 220. In the alternative, handheld unit 104 may be configured such that relay logger unit controller 108 can directly activate heaters 221 and 222 via bus 102.

It should be understood for those skilled in the art that while the electrochemical sensing performed by the fuel cell is the most common way to detect the breath alcohol content in human breath, there are other sensors that can be used. These sensors include semiconductor sensors, light/spectrometry based sensors and others. Performance of all of these substance sensors is affected by their temperature. Hence, the principles of the present invention are not limited to substance testing devices using fuel cells, but are equally application to systems using other types of substance sensors, as well. Generally, the inventive principles are applicable to all devices equipped with substance sensors that require operation at a given temperature or temperature range, be it for reliability, test repeatability, measurement accuracy or any other reason.

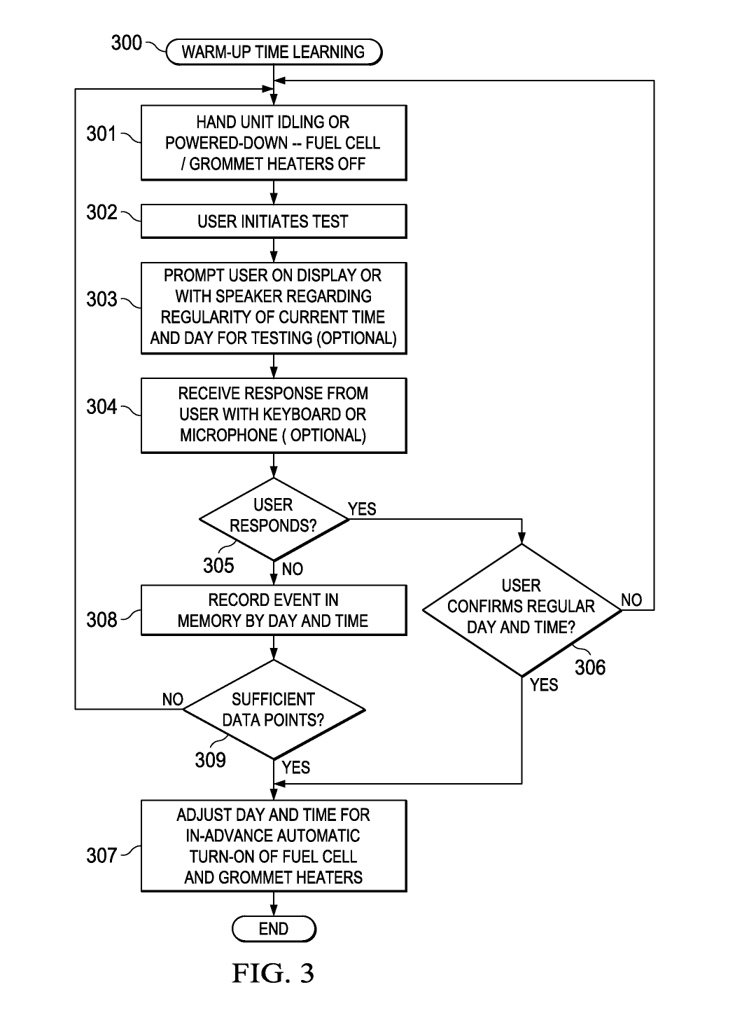

FIG. 3 is a flow chart illustrating an exemplary procedure 300 for training a sobriety system to automatically warm-up in advance of a test, which is expected based on prior user behavior. It should be recognized that alternate training procedures may be used in actual practice and/or alternate embodiments.

At Block 301, handheld unit 100 is either idling (e.g., in an embodiment in which handheld unit controller 104 is directly controlling the warm-up operations) or is powered-down (e.g., both relay/logger unit controller 108 and handheld unit controller 104 are controlling warm-up). In both cases, fuel cell heater 221 and grommet heater 222 are deactivated.

For discussion purposes, it will be assumed that the system has not been trained (programmed) for automatic warm-up for the current day and time on which the user is now requesting a test. Therefore, at Block 302, the user initiates the test in the typical fashion, for example, using keypad 105 and display 106 or by simply activating the vehicle ignition system.

Optionally, the user is prompted using display 106 or speaker 214 to indicate whether testing at the current day and time is expected to be a regular occurrence (Block 303). The user can then respond using keyboard 105 or microphone 213 at Block 304. If the user actively responds at Block 305, and indicates that the current day and time do not represent a regular occurrence (Block 306), then the current day and time or not logged for training purposes and Procedure 300 returns to Block 301 and waits for the next test. In other words, optional Blocks 303-306 advantageously allow for the system to discard data related to use of the vehicle at irregular days and times (e.g., trips from the home or office that are not regularly made at a given time or on a given day.) In addition to random vehicle usage, the user can also confirm regular non-usage of the vehicle. For example, the user can confirm that early morning usage of the vehicle on the weekends is not a regular event and regular scheduling of the warm-up of the testing system is therefore not required. On the other hand, if the user confirms at Block 306 that the current day and time are typical of the user's behavior, then Procedure 300 jumps to Block 308 and the day and time are programmed into memory. Next, in Block 309 the amount and consistency of data is evaluated and when the data is deemed insufficient, the method returns to Block 301. When the data is deemed sufficiently complete for scheduling, the method proceeds to Block 307 and advance warm-up is scheduled. For example, if the user regularly departs for work on Mondays at 7:30 AM (nominally), then that time and day is directly scheduled. Similarly, if the same departure time is used every week day, then warm-up can be scheduled for every week day at the same time. (A series of prompts provided through display 106 and/or speaker 214 advantageously allow the user to flexibly schedule warm-up times based on expected periodic behavior.)

In the preferred embodiment, the algorithms implemented at Block 307 are adaptive, which advantageously accounts for the fact that no person's behavior perfectly follows a pattern. For example, a given user may nominally start the vehicle at around 7:30 AM every weekday, although on some weekdays the user may actually attempt to start the vehicle at 7:35 AM and on other weekdays, the user may actually attempt to start the vehicle at 7:25 AM. In this case, the algorithm, in response to the recorded statistics, adapts the warm-up procedure to have the fuel cell assembly warmed-up and operable by 7:25 AM each weekday (e.g., the earliest time in the window with a statistically sufficient number of occurrences). In other words, the algorithm adapts to provide a window to account for small variations in daily user behavior.

In addition, a user's behavior (e.g., habits) may also change with time. For example, a user may nominally start the vehicle around 7:30 AM on weekdays during October, at nominally 7:45 AM during November, and at nominally 8:00 AM during December. To account for such changes in behavior over time, the adaptive algorithm continually observes statistics over a window of time (e.g., 2 to 4 weeks) and adjusts the fuel assembly warm-up start time accordingly.

If the user does not respond at Block 305, or if the particular embodiment does not provide for the prompting and response steps of Blocks 303-306, then at Block 308, the day and time of the test are recorded in memory. Once there are enough data points to statistically establish with a sufficient degree of confidence that the recorded day and time represents a regular occurrence (Block 309), then Procedure 300 jumps again to Block 307 and the day and time are scheduled for advance warm-up. Otherwise, Procedure 300 returns to Block 301 and waits for the next test.

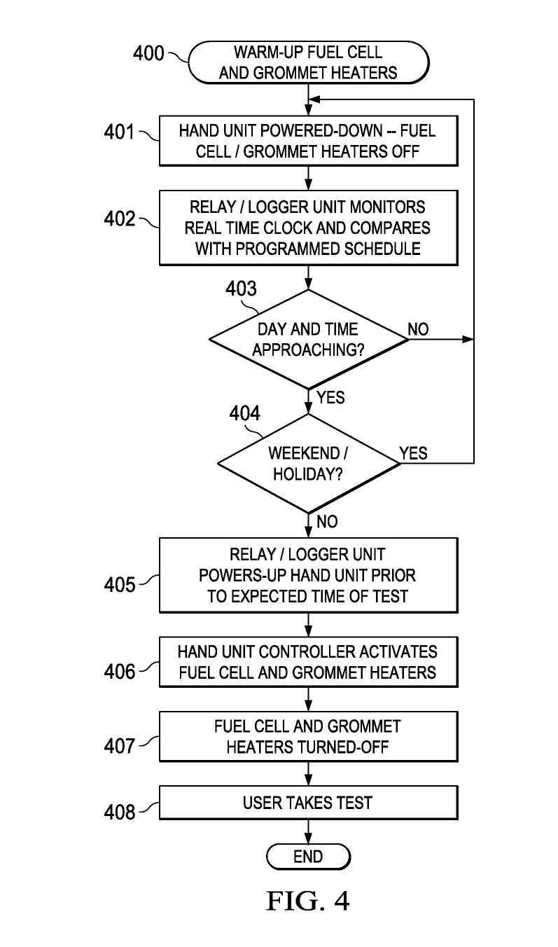

FIG. 4 is a flow chart of a representative procedure 400 for warming-up fuel cell 208 and grommet 200 in advance of an expected test. Procedure 400 is particularly advantageous for an embodiment using both relay/logger unit controller 108 and handheld unit controller 104 to control warm-up, but is not specifically limited thereto.

At Block 401, handheld unit 100 is powered-down and fuel cell heater 221 and grommet heater 222 are off. Relay/logger unit controller 108 monitors real time clock 117 (or the internal clock reference) and compares the current time and day with the programmed schedule (Block 402). If the programmed day and time are approaching, at Block 403, then, optionally, a determination is made at Block 404 as to whether the day falls on a weekend, holiday, or another date on which the user's regular schedule might be expected to vary, or simply that the warmup time is not scheduled for this day. If so, then Procedure 400 returns to the idle state at Block 401. Otherwise, at Block 405, relay/logger unit controller 108 powers-up handheld unit 100 with sufficient time in advance of the expected test to allow heaters 221 and 222 to bring fuel cell 208 and grommet 200 to the appropriate operating temperature. The time required to warm up may very depending on the temperature of the unit and the ambient temperature and humidity outside of the unit. At Block 406, handheld unit controller 104 activates fuel cell heater 221 and grommet heater 222. Subsequently, the user takes the test (Block 407) and fuel cell heater 221 and grommet heater 222 are shut off (Block 408).

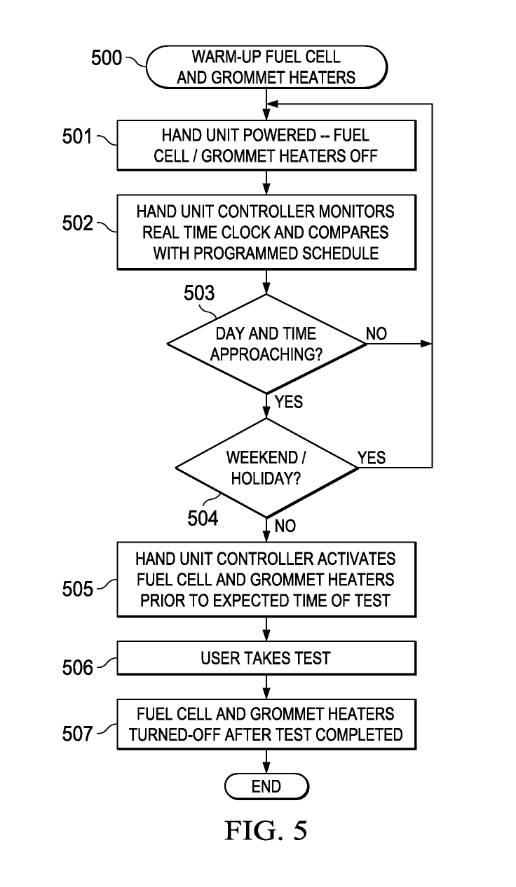

FIG. 5 is a flow chart of another representative procedure 500 for warming-up fuel cell 208 and grommet 200 in advance of an expected test. Procedure 500 is particularly advantageous for an embodiment in which handheld unit controller 104 remains at least partially powered while the vehicle is turned-off and directly controls fuel cell and grommet warm-up.

At Block 501, handheld unit 100 is at least partially powered, but fuel cell heater 221 and grommet heater 222 are inactive. Handheld unit controller 104 monitors the real time clock 223 (and/or internal clock reference) and compares the current time and day with the programmed schedule (Block 502).

If the programmed day and time are approaching, at Block 503, then, optionally, a determination is made at Block 504 as to whether the day falls on a weekend, holiday, or another date on which the user's regular schedule might be expected to vary, or simply that the warmup time is not scheduled for this day. If so, then Procedure 500 returns to the idle state at Block 501.

Otherwise, at Block 505, handheld unit controller 104 activates fuel cell heater 221 and grommet heater 222 with sufficient time in advance of the expected test to allow heaters 221 and 222 to bring fuel cell 208 and grommet 200 to the appropriate operating temperature. Subsequently, the user takes the test (Block 506) and fuel cell heater 221 and grommet heater 222 are shut off (Block 507).

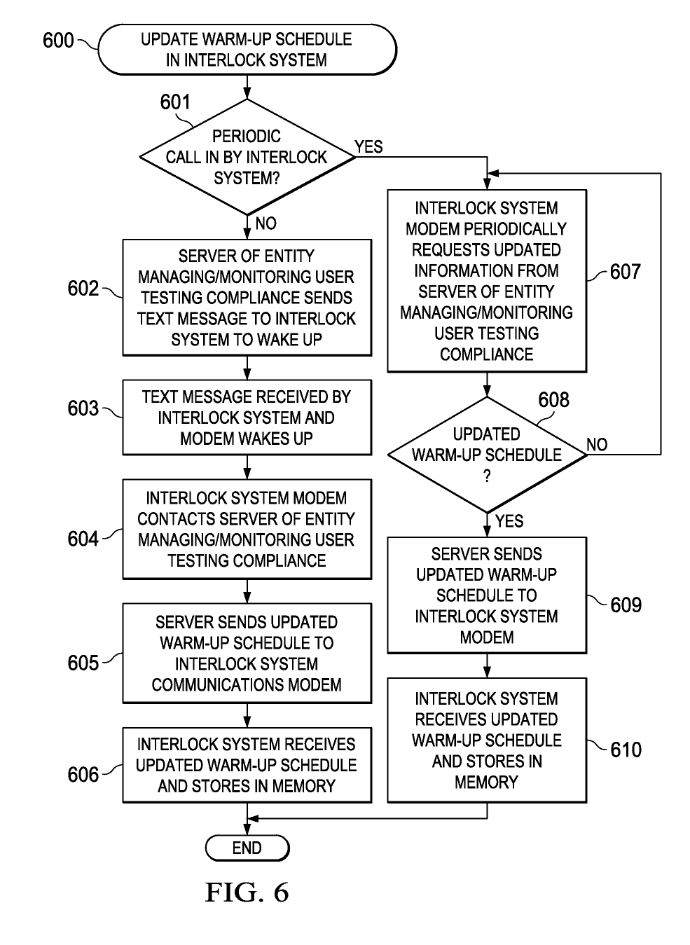

FIG. 6 is a flow chart illustrating an exemplary procedure 600 for updating the warm-up schedule programmed into relay/logger unit memory 107 from the server of the entity managing and/or monitoring user compliance. Procedure 600 allows update the warm-up schedule either by periodic call-ins from the interlock system to the server, in response to text messaging (e.g., SMS) from the server to the interlock system, or both (Block 601).

In first operating mode, the interlock system is either not periodically contacting the server for updates to the warm-up schedule or is periodically contacting the server, but the server nevertheless must send an update in advance of the next periodic call from the interlock system (e.g., the server needs to effectuate an early, urgent, or unscheduled update.) At Block 602, the server sends a text message to the interlock system to wake-up communications modem 114 (FIG. 1B). Communications modem 114 subsequently wakes-up at Block 603 and the interlock systems responds to the server at Block 604.

Once the server and communications modem 114 have established communications, the server sends the updated warm-up schedule to communications modem 114 (Block 605). At Block 606, relay/logger unit controller 108 takes the updated warm-up schedule received through communications modem 114 and stores it in relay/logger unit memory 107 or any other memory associated with the interlock system.

In the second mode of operation, the interlock system periodically contacts the server using communications modem 114 (Block 607) for warm-up schedule updates. (Although as discussed above, the server is still able to send text messages to the interlock system for early, unscheduled, or urgent updates).

When an updated warm-up schedule is available on the server at Block 608, the server sends the updated information to communications modem 114 of the interlock system (Block 609). At Block 610, relay/logger unit controller 108 takes the updated warm-up schedule received through communications modem 114 and stores it in relay/logger unit memory 107 or any other memory associated with the interlock system.

In addition to receiving warm-up schedule updates from a server, alternate embodiments of the present invention allow the user to directly input those updates through keypad 105 and display 106 of hand-held unit 100 (FIG. 1B) or by directly sending a text to communications modem 114, which in the preferred embodiment has the capability of parsing text messages and send the extracting information to relay/logger unit controller 108 to program relay/logger unit memory 107 with the updated warm-up schedule.

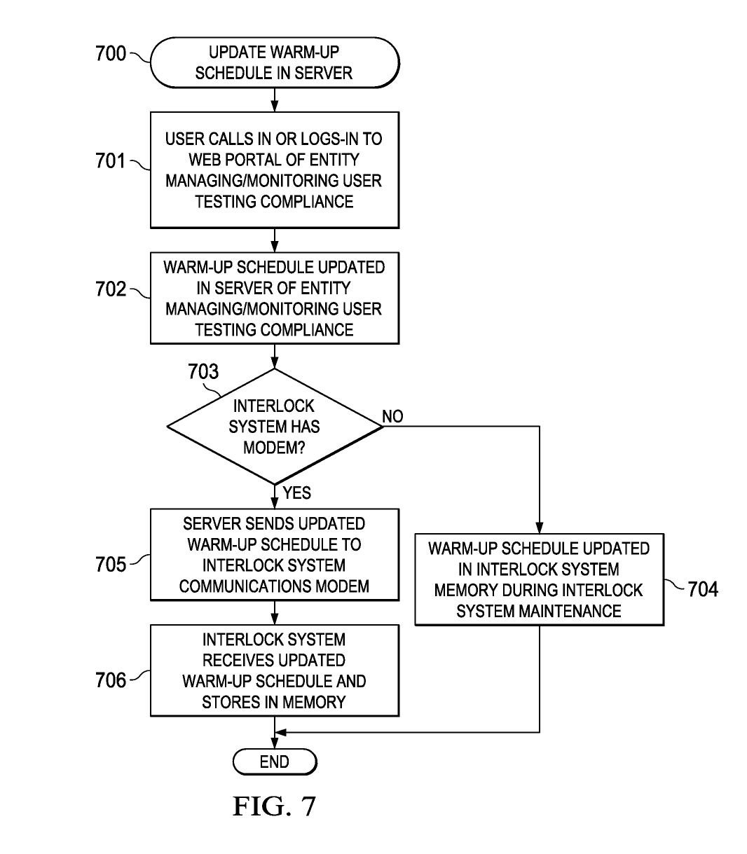

An exemplary procedure 700 for updating the warm-up schedule on the server of the entity managing and/or monitoring user compliance is shown in the flow chart of FIG. 7. In addition, procedure 700 addresses the situation where the interlock system does not include communications modem 114 or similar device supporting remote (wireless) updates.

At Block 701, the user either calls into to the entity managing and/or monitoring user compliance by telephone or logs into that entity's web portal. Alternatively, the user may use a smart phone or computer application software, which directly communicates with the server of the entity managing and/or monitoring user compliance.

At Block 702, the updated warm-up schedule is entered into the server, either directly by the user (i.e., through the website or computer or smart phone) or by personnel communicating with the user in person or by telephone. If the user's interlock system does not include communications modem 114 or a similar device (Block 703), then warm-up schedule update must be performed during periodic (e.g., monthly) maintenance of the interlock system by the entity managing and/or monitoring user compliance (Block 704). The update could, for example, be implemented by a physical transfer to the interlock system, such as through a cable, portable memory stick, or updated memory chips.

Otherwise, procedure 600 discussed above is preferably used to implement the update. However, generally, the server sends the updated warm-up schedule to communications modem 114 of the interlock system (Block 705) and relay/logger unit controller 108 stores the updated schedule in memory (Block 706).

Although the invention has been described with reference to specific embodiments, these descriptions are not meant to be construed in a limiting sense. Various modifications of the disclosed embodiments, as well as alternative embodiments of the invention, will become apparent to persons skilled in the art upon reference to the description of the invention. It should be appreciated by those skilled in the art that the conception and the specific embodiment disclosed might be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore contemplated that the claims will cover any such modifications or embodiments that fall within the true scope of the invention.

What is claimed is:

1. A subsystem for warming-up a substance sensor within a sobriety testing system comprising:
a memory storing a schedule for tests expected to be taken by a user; and
processing circuitry responsive to the stored schedule for activating a heater associated with the fuel cell sufficiently in advance of an upcoming expected test such that the fuel cell is at operating temperature when the upcoming expected test is requested by at the user.

2. The subsystem of claim 1, wherein the memory stores a schedule scheduling each test by expected day and time.

3. The subsystem of claim 1, wherein the sobriety testing system further comprises a grommet for passing breath air from the user to the substance sensor and the processing circuitry is further operable to activate a heater associated with the grommet in advance of the upcoming expected test such that the grommet is at operating temperature when the upcoming expected test is requested by the user.

4. The subsystem of claim 1, further comprising circuitry for allowing the user to directly program a test into the schedule stored in the memory.

5. The subsystem of claim 1, further comprising processing circuitry for automatically programming a test into the schedule stored in the memory in response to statistics taken of user behavior.

6. The subsystem of claim 1, wherein substance sensor forms a portion of a handheld testing unit adapted for use within a vehicle passenger compartment and the processing circuitry comprises a controller disposed within the handheld unit and responsive to the schedule stored in memory for activating the heater sufficiently in advance of the upcoming expected test such that the substance sensor is at operating temperature when the upcoming expected test is requested by at the user.

7. The subsystem of claim 1, wherein the substance sensor forms a portion of a handheld testing unit adapted for use within a vehicle passenger compartment and the processing circuitry comprises a controller in electrical communication with the handheld testing unit and separated from the vehicle passenger compartment by a dashboard, the controller responsive to the schedule stored in memory for activating the heater sufficiently in advance of an upcoming expected test such that the substance sensor is at operating temperature when the upcoming expected test is requested by at the user.

8. The subsystem of claim 1, wherein the processing circuitry comprises:
a first controller forming a portion of a handheld unit adapted for use within a vehicle passenger compartment and including the substance sensor, the first controller providing a signal for activating the heater; and
a second controller operating in conjunction with the controller, wherein the second controller in response to the schedule stored in memory provides power to the first controller to allow the first controller to activate the heater sufficiently in advance of an upcoming expected test such that the substance sensor is at operating temperature when the upcoming expected test is requested by the user.

9. A method of programming an automatic warm-up schedule within a memory of a sobriety testing system comprising:
receiving a request for a test by a user;
recording a day and time for the request for a test;
determining if sufficient data points representing requests for a test at about the same day and time have been previously recorded; and
if sufficient data points representing requests for a test at about the same day and time have been previously recorded, programming the memory to include the day and time in the schedule for automatic warm-up of at least one sub-system within the sobriety testing system.

10. The method of claim 9, further comprising:
in response to receiving the request for a test, prompting the user to enter information indicating the regularity of the day and time for a request for test; and
in response to information entered by the user in response to the prompt indicating that the day and time is a regular day and time for a request for test, programming the memory to include the day and time in the schedule for automatic warm-up of at least one subsystem within the sobriety testing system.

12. The method of claim 9, further comprising programming the memory to include selected days and times at which automatic warm-up is not performed.

13. The method of claim 12, wherein the selected days are selected from the group consisting of weekends and holidays.

14. A method of programming an automatic warm-up schedule within a memory of a sobriety testing system comprising:
storing a warm-up schedule on a server;
updating the warm-up schedule on the server;
transferring an updated warm-up schedule from the server to the memory of the sobriety testing system.

15. The method of claim 14, wherein transferring the updated warm-up schedule to the memory of the sobriety testing system comprises:
sending a text message from the server to the sobriety testing system;
in response to receiving the text message at the sobriety testing system, establishing a wireless communications link between the server and the sobriety testing system;
transmitting the updated warm-up schedule from the server to the sobriety testing system via the wireless link; and
storing the updated warm-up schedule in the memory of the sobriety testing system.

16. The method of claim 14, wherein transferring the updated warm-up schedule to the memory of the sobriety testing system comprises:
periodically contacting the server with the sobriety testing system via a wireless link;
in response to receipt by the server of a periodic contact from the interlock system when an updated warm-up schedule is available, transmitting the updated warm-up schedule from the server to the sobriety testing system via the wireless link; and
storing the updated warm-up schedule in the memory of the sobriety testing system.

17. The method of claim 14, wherein transferring the updated warm-up schedule to the memory of the sobriety testing system comprises a physical transfer.

18. The method of claim 14, wherein updating the warm-up schedule on the server comprises receiving information from a user of the sobriety testing system via a web portal.

19. The method of claim 14, wherein updating the warm-up schedule on the server comprises inputting information received from a user of the sobriety testing system via telephone.

20. The method of claim 14, wherein updating the warm-up schedule on the server comprises receiving information from a user of the sobriety testing system via a software application communicating with the server.
